(19) United States
(12) Patent Application Publication
(10) Pub. No.: US 2010/0239535 A1
(43) Pub. Date: Sep. 23, 2010

(54) METHOD FOR ISOLATING HOMOGENEOUS POPULATIONS OF TRANSDUCED PROGENITORS STABLY EXPRESSING SPECIFIC LEVELS OF A TRANSGENE

(75) Inventors: Andrea Banfi, Basel (CH); Heidi Misteli, Basel (CH)

Correspondence Address: WENDEROTH, LIND & PONACK, L.L.P., 1030 15th Street, N.W.,, Suite 400 East, Washington, DC, 20005-1503, US

(73) Assignee: UNIVERSITY OF BASEL, Basel (CH)

(21) Appl. No.: 12/227,291

(22) PCT Filed: May 16, 2007
(86) PCT No.: PCT/EP2007/054743

§ 371 Date: Nov. 13, 2008

Related U.S. Application Data
(60) Provisional application No. 60/747,426, filed on May 17, 2006.

(51) Int. Cl. A61K 35/00; G01N 33/53; C12N 15/74; A61P 43/00

(52) U.S. Cl. 424/93.2; 435/7.92; 435/7.2; 435/7.21; 435/320.1

(57) ABSTRACT

The invention is directed to a method for producing precursor cells expressing a target gene, for example VEGF, at desired levels comprising stably transducing precursor cells with the target gene linked to a cell-surface marker gene, for example truncated CD8/CD8a, via an IRES sequence accommodating translation of the target and the marker genes from the same mRNA, then correlating an expression level of the target gene with that of the cell-surface marker gene, selecting a reference clone of cultured precursor cells expressing the target gene at the desired threshold, and isolating precursor cells having the same or similar level of expression of the target gene than the reference clone.

### FIELD OF THE INVENTION

The invention relates to a method for rapidly isolating homogeneous populations of transduced progenitors stably expressing specific levels of a transgene by FACS, useful for cell-based gene therapy.

### BACKGROUND OF THE INVENTION

Vascular Endothelial Growth Factor (VEGF) is an attractive candidate being investigated to achieve therapeutic angiogenesis in ischemic diseases, but it also can induce aberrant vasculature and angioma growth, which can call into question its therapeutic utility (M. L. Springer et al., Mol. Cell 2:549-558, 1998; P. Carmeliet, Nat. Med. 6:1102-1103, 2000; S. Ylä-Herttuala et al., J. Am. Coll. Cardiol. 49:1015-1026, 2007). It was recently shown that a discrete threshold in VEGF dosage exists, below which normal stable capillaries are induced and above which angioma growth occurs. However, to predictably avoid toxic effects, it is necessary to ensure homogeneous expression in the microenvironment around each transduced muscle fiber (C. R. Ozawa, A. Banfi et al., J. Clin. Invest. 113:515-527, 2004). In fact, since VEGF remains tightly localized in tissue around the cells producing it, different growth factor concentrations do not average each other, even between neighbouring muscle fibers. Therefore, a few “hotspots” of high expression are sufficient to cause hemangioma growth even if the total VEGF dose is rather low. This finding helps to explain the apparent difficulty to achieve a manageable therapeutic window in clinical trials of VEGF gene therapy (A. Banfi et al., Curr. Atheroscl. Rep. 7:227-234, 2005). In fact, currently employed gene therapy methods, such as direct injection of constitutive adenoviral and plasmid vectors, only allow control of the total dose (titer) of gene delivered, but not of the distribution of microenvironmental levels achieved in vivo. Therefore, in order to avoid even rare “hotspots” of expression, the total dose must be kept low and efficacy is wasted.

### SUMMARY OF THE INVENTION

The present invention is directed at a method for producing precursor cells expressing a target gene at desired levels, such as efficacious levels, more efficacious levels or microenvironmentally sub-toxic levels, comprising

(a) providing a heterogeneous population of cultured precursor cells;
(b) stably transducing said precursor cells with (i) a target gene linked to (ii) a cell-surface marker gene via a sequence accommodating translation of (i) and (ii) from the same mRNA;
(c) correlating an expression level of the target gene with that of the cell-surface marker gene;
(d) selecting a reference clone of cultured precursor cells expressing said gene at a threshold of desirability, e.g., at a threshold of (i) efficacious expression, (ii) increased efficacious expression or (iii) microenvironmentally sub-toxic expression of said gene; and
(e) isolating precursor cells having the (i) same or lower levels or (ii) the same or higher levels of expression of said target gene than said reference clone via said cell-surface marker.

In a variant of this method, two reference clones are selected in step (d) expressing said gene at a lower threshold and at an upper threshold, respectively, and precursor cells are isolated in step (e) having a level of expression of said target gene between the levels of expression of the two reference clones. In a further variant, two or more, e.g., three, four or five, reference clones are selected in step (d) expressing said gene at different levels, the plurality of clones used to define a lower and an upper threshold of target gene expression, and precursor cells isolated in step (e) having a level of expression of said target gene at any desired level between the lower and higher threshold levels of expression.

The expression level of said cell-surface marker in (d) may be quantified via Fluorescence Activated Cell Sorting (FACS). The expression level of said target gene in (d) may be quantified via methods such as, but not limited to, ELISA, RIA, EIA, Western Blot or Surface Plasmon Resonance.

The target gene may be a Vascular Endothelial Growth Factor (VEGF) or any other gene, such as, but not limited to, a gene encoding a transcription factor or a growth factor receptor, having a toxic effect when expressed in a host microenvironment in excess of a determined threshold level or for which a minimum expression level is desired for efficacy or for increased efficacy, such as, but not limited to, constitutively active HIF-1 alpha, PDGFR beta or PDGFb.

Said cell-surface marker may be any endogenous cell-surface compound readily quantifiable, preferably in truncated form without the original biological activity, for example, a truncated version of CD8/CD8a.

The sequence accommodating translation of (b)(i) and (b)(ii) from the same mRNA may be an internal ribosomal entry site (IRES).

Said precursor cells may be primary myoblasts, bone marrow derived mesenchymal stem cells, adipose tissue-derived mesenchymal stem cells, or cardiac stem cells.

The invention is also directed towards a system for producing precursor cells expressing a target gene in a host at microenvironmentally desirable levels, e.g., sub-toxic levels comprising

(a) a construct integrating into the host genome, such as, but not limited to, a retroviral construct, providing (i) a target gene linked to (ii) a cell-surface marker via a sequence accommodating translation of (i) and (ii) from the same mRNA; and
(b) instructions for:
(1) stably transducing precursor cells with said construct; and
(2) correlating expression levels of the target gene with that of the cell-surface marker so that expression levels of said target gene can be assessed in said precursor cells via said cell surface marker.

The invention is also directed towards a system for in vivo gene therapy comprising a homogenous population of precursor cells stably transduced with a construct providing (i) a target gene linked to (ii) a cell-surface marker via a sequence accommodating translation of (i) and (ii) from the same mRNA, wherein the homogenous population has been selected via said cell surface marker so that the expression levels of said target gene are in the desired range, e.g., efficacious, more efficacious or microenvironmentally sub-toxic, in a host microenvironment into which said precursor cells are to be introduced.

The invention is also directed towards a controlled level expression gene therapy comprising

(a) obtaining a heterogeneous population of precursor cells from a patient;
(b) culturing and stably transducing said precursor cells with (i) a target gene linked to (ii) a cell-surface marker via a sequence accommodating translation of (i) and (ii) from the same mRNA;
(c) correlating in clones of said precursor cells expression levels of the target gene with that of the cell-surface marker gene;
(d) selecting, as a reference clone, a clone of (c) expressing said target gene at a level that defines a threshold of desirability for said patient, e.g., of (i) sub-toxicity or (ii) a higher level of efficacy;
(e) isolating via said cell-surface marker cells having the (i) same or lower levels or (ii) the same or higher levels of expression of said target gene than said reference clone; and
(f) reintroducing the cells of (e) into said patient.

In a variant of this method, two reference clones are selected in step (d) expressing said gene at a lower threshold and at an upper threshold, respectively, and precursor cells are isolated in step (e) having a level of expression of said target gene between the levels of expression of the two reference clones.

Furthermore, in a controlled level expression gene therapy said steps (c) and (d) may be omitted and the information obtained in steps (c) and (d) from the application of the method on precursor cells of another patient be used in place thereof, i.e. the correlation of expression levels of the target gene with that of the cell-surface marker gene and the selection of a reference clone.

### BRIEF DESCRIPTION OF THE FIGURES

FIG. 1. Control over microenvironmental distribution enhances the efficacy of VEGF gene delivery in ischemic muscle.

Vehicle only (BSA), control cells (LacZ) or myoblast populations expressing the indicated VEGF levels (5, 60 or 100 ng/10⁶ cells/day) were implanted in ischemic mouse hind limbs. Analysis after 2 weeks shows that homogeneous distribution of expression levels in the microenvironment, achieved by a clonal population (C), significantly improves both relative blood flow (a.) and number of collateral arteries (b.) compared to the same total dose (60 ng/10⁶ cells/day) delivered as the average of dishomogeneous levels by a polyclonal population (PC).

R.B.F. (%)=Relative blood flow (% of non-ischemic). No. Coll.=Number of collaterals. PC=polyclonal, C=clonal.

FIG. 2. Structure of the VICD8 retroviral construct.

Expression of the mouse VEGF₁₆₄ gene (mVEGF₁₆₄) was linked to that of the FACS-sortable cell-surface marker truncated CD8a (tr. mCD8a) through an IRES sequence. This cassette was inserted into a retroviral construct, so that stable expression of the two genes is achieved after integration into the genome of the target cell.

LTR=retroviral long terminal repeats; ψ=retroviral packaging signal.

FIG. 3. Correlation of VEGF production with amount of fluorescence in standard populations.

Individual clones homogeneously expressing different levels of VEGF were analyzed on the FACS after staining with a fluorescently-labeled antibody against mCD8a. The intensity of CD8 staining and the production of VEGF (measured by ELISA) were found to correlate with a value of R²=0.8969.

X-axis: VEGF production (ng/10⁶ cells/day), Y-axis: CD8 expression (fluorescence intensity units).

FIG. 4. FACS-sorting of desired populations based on CD8 expression level.

Almost all cells in the primary transduced population express CD8 (and VEGF) compared to the negative control (below 2×10¹), but the spectrum of expression levels is widely heterogeneous (between 2×10¹ and 10⁴). Based on the range of fluorescence detected in the reference clone (curve between 10² and 10³ reaching 200 counts), two different polyclonal sub-populations have been sorted, expressing lower (A.) or higher (B.) CD8 levels. The purity of the sorted populations was determined by re-analysis on the FACS (dark plots in panels b. and c.) and they were found to be completely contained within the respective sorting gates.

X-axis: CD8 expression (fluorescence intensity units), Y-axis: cell counts.

FIG. 5. Stability of expression of CD8 and of VEGF in sorted populations during in vitro expansion.

The sorted populations described in FIG. 4 (A. and B.) were expanded for about 25 population doublings in vitro. The expression of CD8a (measured by FACS, panel a.) and of VEGF (measured by ELISA, panel b.) was found to be stable, with similar values immediately after sorting and after about 1×10⁷-fold expansion (23 doublings).

Db=population doublings; CD8=CD8 expression (fluorescence intensity units); VEGF=VEGF production (ng/10⁶ cells/day)

FIG. 6. In vivo angiogenesis after implantation of purified VEGF-expressing myoblasts. Cells isolated from a heterogeneous population based on their CD8 expression (A. and B. gates in FIG. 4a.) induce normal angiogenesis (S1 and S2), similarly to the clone used as a reference to sort them (Ref.), but bypassing the cloning step. The unsorted population (N.S.) causes the widespread growth of hemangiomas instead. In the top panels vasculature was stained with lectin and implanted myoblasts were stained for LacZ expression using the X-gal reaction. In the lower panels, the vessel diameter (Dia. (μm)) distribution was quantified in the implantation areas and is expressed as the percentage of vessels (%) with a given diameter in micrometers (μm), rounded to the nearest micrometer.

C-=control cells; N.S.=not sorted population; Ref.=reference clone;

S1=sorted with gate A. (see FIG. 4); S2=sorted with gate B. (see FIG. 4)

FIG. 7. Morphological characterization of induced angiogenesis.

Rapidly purified populations induce a doubling of the vessel length density (VLD) and an increase of the branching index (BA.) relative to controls, similarly to the reference clone, whereas the unsorted population actually reduces the number of vessels in the implantation areas.

VLD=Vessel Length Density (mm of vessel length/mm²); B.I.=Branching Index (number of branch points/100 μm of vessel length);

C-=control cells; N. S.=not sorted population; Ref.=reference clone;

S1=sorted with gate A.; S2=sorted with gate B.

### DETAILED DESCRIPTION OF THE INVENTION

The terms used hereinabove and hereinbelow have the following meaning:

Precursor cells—are cells capable of giving rise to a differentiated progeny in vivo, after in vitro expansion and/or genetic modification. Examples include myoblasts and muscle satellite cells, cardiac progenitors, bone-marrow or adipose tissue-derived mesenchymal progenitor cells (also called mesenchymal stem cells), multipotent adult progenitor cells (MAPC), neural stem cells, epidermal stem cells, corneal stem cells and conjunctival stem cells.
Microenvironmental—refers to the level of expression of a transgene in each individual transduced cell, which determines the amount of gene product that accumulates in the microenvironment around it in vivo, as opposed to the average expression by the total population. The two expressions (total and microenvironmental) coincide when the population is the progeny of a single clone, in which every cell produces the same level.
Sub-toxic—refers to no toxicity or very low toxicity, not causing any persistent damage. In the context of target genes supporting angiogenesis sub-toxic is defined as inducing non-aberrant angiogenesis. This may further be defined as the growth of new blood vessels with little or no detectable formation of defective vascular structures, which differ in structure and/or function from normal capillaries, arteries or veins, specifically including glomeruloid, bulbous or angioma-like structures, hemangiomas, and chronically hyperpermeable vessels.
Heterogeneous population—is a population of transduced cells, composed of several different clonal subpopulations, each of which expresses a different level of the transgene over a wide range of values.
Target gene—is a gene whose gene product requires tight expression control in individual cells transduced with its coding sequences, in order to achieve a desired functional effect, while avoiding toxic effects. Examples include: (1) secreted growth factors/cytokines; (2) membrane and intracellular receptors; and (3) transcription factors. As relating to the process of angiogenesis, examples of each category may include: (1) members of the VEGF/PIGF, PDGF, TGF-beta, or Angiopoietin families; (2) their respective receptors; (3) naturally-occurring or modified, constitutively active Hypoxia Inducible Factor (HIF) molecules (K. A. Vincent et al., Circulation 102:2255-2261, 2000), or angiogenic factors-activating engineered transcription factors (E. J. Rebar et al., Nat. Med. 8:1427-1432, 2002).
Cell-surface marker—is a protein which: (1) is located on the cell surface, so that it can be quantified by antibody staining and FACS detection; (2) is the product of a human gene, so that it does not elicit an immune reaction; and (3) does not have a biological function in the stably transduced cells, so that its expression does not interfere with cell expansion in vitro and differentiation in vivo. Examples include the lymphocite-specific molecules CD8 and CD4, whose required partners in the T-Cell Receptor Complex are only expressed in T-lymphocites. To further avoid any biological function, said molecules are modified to truncate them at the junction between the transmembrane and intracytoplasmic domains, so that the intracellular signal-transduction domains are removed, while the transmembrane and extracellular domains, which are responsible for cell-surface localization and antibody recognition, respectively, are retained.

Sequence accommodating translation—is a polynucleotide sequence, positioned between a first and a second coding sequence, which allows translation of both coding sequences from the same transcription unit (a single mRNA molecule). Examples include a variety of naturally-occurring or synthetic Internal Ribosomal Entry Sites (IRES), such as the Encephalomyocarditis virus IRES described in the experimental part hereinbelow. Alternatively, IRES sequences derived from rhinovirus, aphthovirus, cardiovirus, encephalomyocarditis virus, enterovirus, adenovirus, influenza virus, herpes virus cytomegalovirus, HIV, mengovirus, bovine viral diarrhea virus, hepatitis A, B or C virus, GTX, Cyr61a, Cyr61b, poliovirus, picornavirus, murine encephalomyocarditis virus, poliovirus, and foot and mouth disease virus may be used. Fragments, mutants and variants of naturally occurring IRES sequences may be employed provided they retain the ability to initiate translation of an operably linked coding sequence located 3′ of the IRES.

Correlate—means to establish a quantitative relationship between the levels of expression of the target gene and of the cell-surface marker in each transduced cell, by measuring each in clonal populations (in which each cell expresses the same level), so that measurement of the second may give information on the value of the first in individual cells in heterogeneous populations.

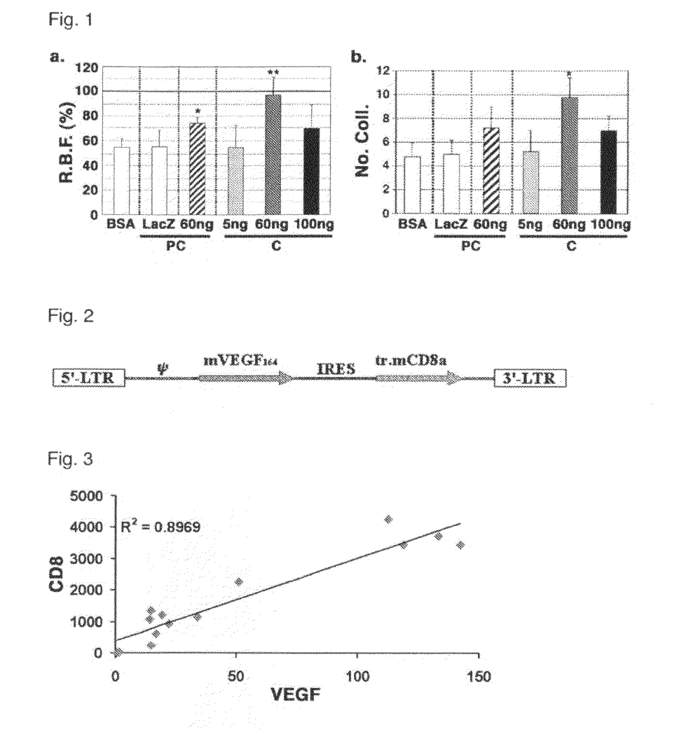

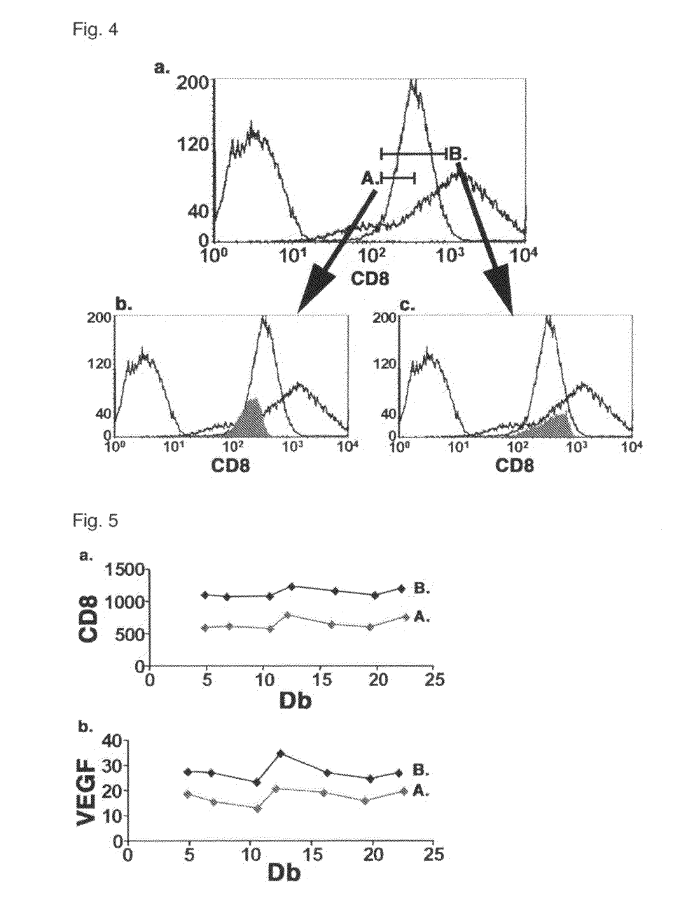

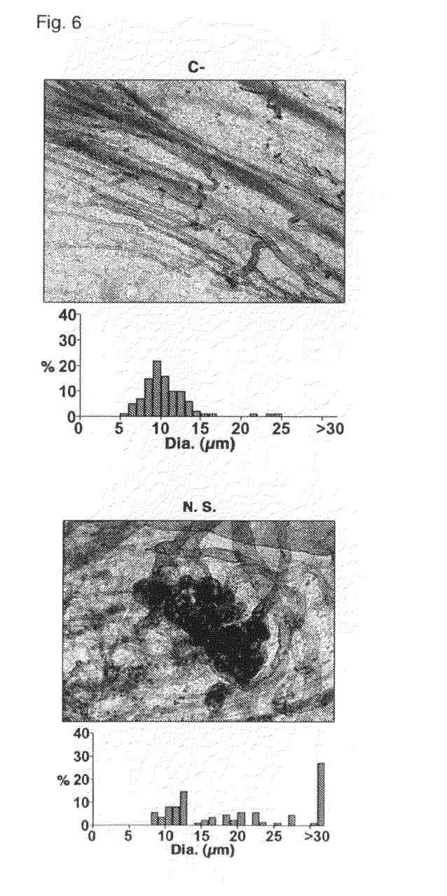

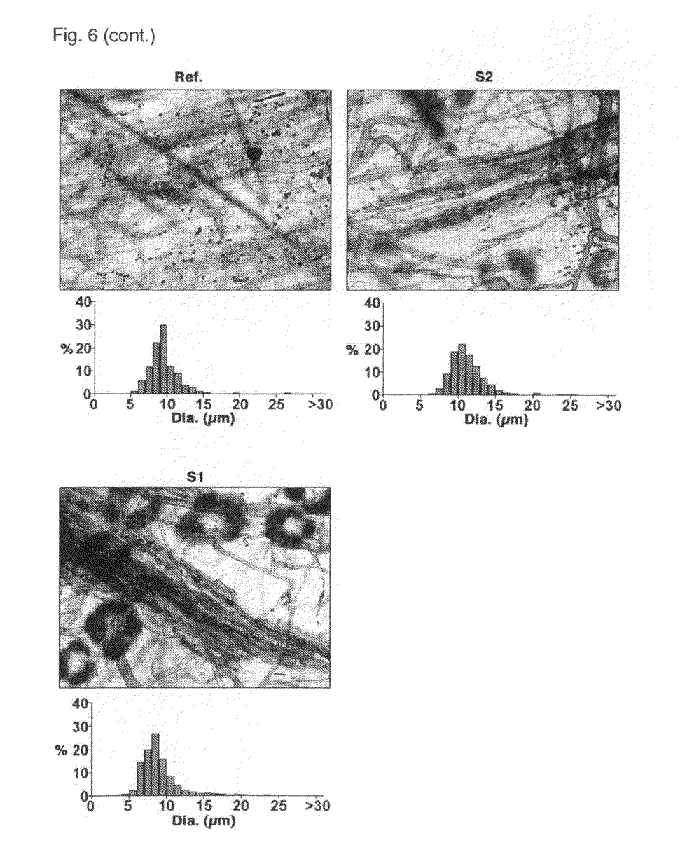

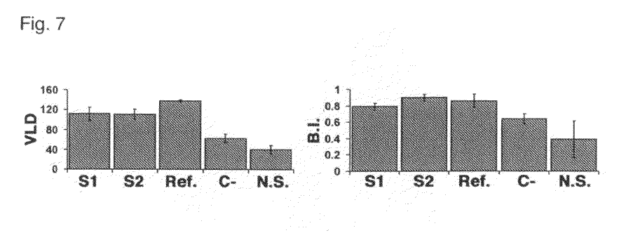

The rationale for the inventive method is the following: The distribution of microenvironmental expression levels can be reliably controlled by implanting clonal myoblast populations stably expressing different homogeneous levels of VEGF per cell (C. R. Ozawa, A. Banfi et al., J. Clin. Invest. 113:515-527, 2004). The therapeutic potential of controlling the microenvironmental distribution of VEGF levels is shown in a murine model of hindlimb ischemia. Implantation of a polyclonal myoblast population, expressing VEGF₁₆₄ at 60 ng/10⁶ cells/day on average, increases blood flow only moderately (FIG. 1a), and vascular leakage and aberrant pre-angiomatous vessels are always induced. However, when the same total VEGF dose is uniformly distributed in muscle by implanting a monoclonal population, blood flow is fully restored to nonischemic levels (FIG. 1a), accompanied by arteriole and collateral growth (FIG. 1b) and reduced ischemic tissue damage. Aberrant vasculature and hemangiomas are avoided and only normal, pericyte-covered vessels are induced which persist over 15 months. While providing compelling proof-of-concept of the therapeutic improvement afforded by VEGF gene delivery at controlled levels, this approach is not feasible in clinical practice. In fact, it involves the isolation and characterization of multiple clonal populations of transduced autologous myoblasts, which requires both excessive time and resources for each patient. To make this concept clinically applicable, it is desirable to be able to rapidly purify cells expressing the desired VEGF level from a randomly transduced autologous population, without producing clones for every patient.

In a particular embodiment of the invention, in order to achieve controlled gene delivery to skeletal muscle, primary myoblasts are transduced with a retroviral construct (mVICD8) in which the VEGF₁₆₄ gene is linked through an Internal Ribosomal Entry Site (IRES) to a truncated version of CD8a (murine Ly2 gene, FIG. 2). The intracellular domain of this lymphocyte-specific molecule is removed, such that it retains no biological function and acts purely as a cell-surface reporter gene. An IRES sequence allows ribosome assembly on the mRNA and the initiation of translation in a cap-independent fashion (I. R. Ghattas et al., Mol. Cell. Biol. 11: 5848-5859, 1991). Therefore it allows the translation of two genes from the same mRNA: The first by the classic mechanism and the second through the IRES. In this way, changes in the level of expression of VEGF are reflected by a parallel change in cell-surface expression of CD8a, which can be detected and quantified on live cells by Fluorescence Activated Cell Sorting (FACS).

The basic concept underlying this embodiment of the invention is that the linking of VEGF expression to that of CD8 on the cell surface allows the prediction of the level of expression of VEGF on a cell-by-cell basis by quantification of the CD8 level by FACS. However, this is not possible directly, because the absolute intensity of fluorescence detected by the FACS depends on many variables, including the efficiency of antibody staining, the laser intensity and the settings of the detectors, among others. Therefore, in order to reproducibly identify the cells expressing a desired VEGF level in the heterogeneous primary transduced population, it is necessary to have a reference population which stably and homogeneously expresses that level and which is stained and analyzed together with the primary cells. The cells which co-localize with the reference population on the FACS plot express equivalent VEGF levels and can be sorted to purity.

Primary mouse myoblasts have been transduced with the mVICD8 retroviral construct, generating a very heterogeneous polyclonal population. In order to establish the relationship between VEGF production and level of CD8 expression, single clones have been isolated from this population, stably expressing different and homogeneous VEGF and CD8 levels (FIG. 3).

A set of 8 clones expressing different levels of VEGF were also injected in vivo in the posterior auricular muscle of immunodeficient SCID mice, as previously described (C. R Ozawa, A. Banfi et al., J. Clin. Invest. 113:515-527, 2004), in order to evaluate the induced vascular phenotype. The population that expressed the highest VEGF amount, while at the same time completely avoiding aberrant angiogenesis (−40 ng/10⁶ cells/day) was selected as the reference clone. The stability of VEGF and CD8 expression by this clone was tested and it was found to be constant over at least 26 population doublings.

As shown in FIG. 4, the reference clone was used to identify 2 different polyclonal sub-populations of interest from the primary heterogeneous population. Based on their level of CD8 expression, one group of cells was sorted corresponding to the whole width of the reference clone (large gate B. in FIG. 4) or just to its lower half (narrow gate A.).

The two sorted populations were cultured in vitro to simulate the expansion necessary to achieve sufficient cells for clinical application. Both CD8 expression and VEGF production (−20 and −30 ng/10⁶ cells/day for the narrow and large populations, respectively) were found to be stable over at least 23 populations doublings (FIG. 5). This degree of expansion would yield over 8×10¹³ cells from an initial sort of 1 million, which is several orders of magnitude in excess of the amount needed for clinical delivery (estimated in the range of 10⁹ cells). When implanted in vivo in the posterior auricular muscle of SCID mice, the sorted populations showed that a highly significant enrichment of the cells expressing the desired VEGF levels had been achieved. As shown in FIG. 6, the heterogeneous polyclonal population invariably induced aberrant bulbous structures, which evolve into progressive cavernous hemangiomas, as expected. However, the cells sorted from this same population based on their CD8 expression (both with the narrow and large gates) caused the growth of normal, homogeneously sized capillaries, similarly to the reference clone. Analysis of the vessel diameter distribution shows that normal capillaries have sizes comprised between 5 and 15 μm, with a peak below 10 μm (FIG. 6, control C-). The bulbous structures induced by heterogeneous high levels of VEGF have aberrantly dilated diameters, often larger than 30 μm (FIG. 6, unsorted N.S.). Both sorted populations induce angiogenesis with a normal diameter distribution, similarly to the reference clone (FIG. 6, S1, S2 and Ref.).

Both sorted populations preserve a normal capillary phenotype, but also efficiently induce new vascular growth. In fact, vessel length density (VLD), which measures the total length of vessels in a defined area and is not influenced by diameter, is doubled in the areas implanted with both sorted populations compared to control (FIG. 7, left panel), similarly to the effects of the reference clone. Interestingly, dishomogeneous and high VEGF levels expressed by the unsorted population actually decrease VLD, because they induce hyperfusion of pre-existing vessels and circumferential growth of bulbous structures, rather than sprouting of new capillaries (FIG. 7, left panel).

This increase in VLD represents an actual increase in vessel number and not simply an elongation of pre-existing capillaries. This is shown by the quantification of the branching index, which measures the number of branch points/100 μm of vessel length. As shown in FIG. 7 (right panel), both sorted populations not only increase VLD compared to control, but also the frequency of branch points. Therefore, the resulting vessels, defined as the vascular segments between 2 branch points, are slightly shorter (more branches) and more than double in number compared to controls. Again, these features are completely similar to the effects of implantation of the reference clone.

At 4 weeks post-implantation in the auricular muscle, only normal angiogenesis had been induced in 4 out of 5 animals injected with each of the 2 sorted populations.

As the person skilled in the art will appreciate, variations of the above described protocols are possible. For example, the sorting gate might assume different sizes (the whole size of the reference clone vs. a tighter gate spanning half the width and centred on the peak) and, apart from a single round of sorting, multiple successive rounds of sorting, such as 2 to 3, may be performed. Furthermore, the center of the sorting gate may be positioned on different expression values, precalculated on the basis of a “reference line” obtained from the values of 2 or more different reference clones, so that populations may be obtained which express any desired level of the target gene within the range of the polyclonal population. Precise definition of the minimum required conditions that reproducibly yield homogeneous safe and efficacious populations is accordingly possible.

It should also be noticed that, while the above-described experiments are performed in the field of therapeutic angiogenesis by VEGF gene delivery, this invention is readily applicable to other genes of interest, in particular those for which it is necessary to tightly control expression levels in each individual transduced cell, such as other secreted growth factors, transcription factors and membrane or intracellular receptors.

1. A method for producing precursor cells expressing a target gene at desired levels comprising
(a) providing a heterogeneous population of cultured precursor cells;
(b) stably transducing said precursor cells with (i) a target gene linked to (ii) a cell-surface marker gene via a sequence accommodating translation of (i) and (ii) from the same mRNA;
(c) correlating an expression level of the target gene with that of the cell-surface marker gene;
(d) selecting a reference clone of cultured precursor cells expressing said target gene at a threshold of desirability; and
(e) isolating precursor cells having the (i) same or lower levels or (ii) the same or higher levels of expression of said target gene than said reference clone via said cell-surface marker.

2. The method of claim 1 wherein in step (d) two reference clones are selected expressing said gene at a lower threshold and at an upper threshold, respectively, and precursor cells are isolated in step (e) having a level of expression of said target gene between the levels of expression of the two reference clones.

3. The method of claim 1 wherein in step (d) two or more reference clones are selected expressing said gene at different levels, said reference clones used to define a lower and an upper threshold of target gene expression, and precursor cells isolated in step (e) having a level of expression of said target gene at any desired level between the lower and higher threshold levels of expression.

4. The method of claim 1 wherein the expression level of said cell-surface marker in step (d) is quantified via Fluorescence Activated Cell Sorting (FACS).

5. The method of claim 1 wherein the expression level of said target gene in step (d) is quantified via a method selected from ELISA, RIA, EIA, Western Blot and Surface Plasmon Resonance.

6. The method of claim 1 wherein the target gene is a gene encoding a transcription factor or a growth factor receptor, having a toxic effect when expressed in a host microenvironment in excess of a determined threshold level or for which a minimum expression level is desired for efficacy or for increased efficacy.

7. The method of claim 6 wherein the target gene is Vascular Endothelial Growth Factor (VEGF).

8. The method of claim 6 wherein the target gene is constitutively active HIF-1 alpha, PDGFR beta or PDGFb.

9. The method of claim 1 wherein the cell-surface marker gene is an endogenous cell-surface compound readily quantifiable, in truncated form without the original biological activity.

10. The method of claim 9 wherein the cell-surface marker gene is a truncated version of CD8/CD8a.

11. The method of claim 1 wherein in step (b) the sequence accommodating translation from the same mRNA is an internal ribosomal entry site (IRES).

12. The method of claim 1 wherein the heterogeneous population of cultured precursor cells is selected from primary myoblasts, bone marrow derived mesenchymal stem cells, adipose tissue-derived mesenchymal stem cells, or cardiac stem cells.

13. A system for producing precursor cells expressing a target gene in a host at desirable levels, comprising
(a) a construct integrating into the host genome providing (i) a target gene linked to (ii) a cell-surface marker via a sequence accommodating translation of (i) and (ii) from the same mRNA; and
(b) instructions for:
(1) stably transducing precursor cells with said construct; and
(2) correlating expression levels of the target gene with that of the cell-surface marker so that expression levels of said target gene can be assessed in said precursor cells via said cell surface marker.

14. A system according to claim 13 wherein the construct is a retroviral construct.

15. A system for in vivo gene therapy comprising a homogenous population of precursor cells stably transduced with a construct providing (i) a target gene linked to (ii) a cell-surface marker via a sequence accommodating translation of (i) and (ii) from the same mRNA, wherein the homogenous population has been selected via said cell surface marker so that the expression levels of said target gene are as desired in a host microenvironment into which said precursor cells are to be introduced.

16. A method of controlled level expression gene therapy comprising
(a) obtaining a heterogeneous population of precursor cells from a patient;
(b) culturing and stably transducing said precursor cells with (i) a target gene linked to (ii) a cell-surface marker via a sequence accommodating translation of (i) and (ii) from the same mRNA;
(c) correlating in clones of said precursor cells expression levels of the target gene with that of the cell-surface marker gene;
(d) selecting, as a reference clone, a clone of (c) expressing said target gene at a level that defines a threshold of desirability for said patient;
(e) isolating via said cell-surface marker cells having the (i) same or lower levels or (ii) the same or higher levels of expression of said target gene than said reference clone; and
(f) reintroducing the cells of (e) into said patient.

17. The method of claim 16 wherein in step (d) two reference clones are selected expressing said gene at a lower threshold and at an upper threshold, respectively, and precursor cells are isolated in step (e) having a level of expression of said target gene between the levels of expression of the two reference clones.

18. The method of claim 16 wherein steps (c) and (d) are omitted and the information obtained in steps (c) and (d) from the application of the method on precursor cells of another patient are used in place thereof.
